(19) United States
(12) Patent Application Publication
(10) Pub. No.: US 2010/0076314 A1
(43) Pub. Date: Mar. 25, 2010

(54) SYSTEM AND METHOD FOR CREATING VIRTUAL FORCE FIELD

(76) Inventors: Robert Muratore, Huntington, NY (US); Sheikh Kaisar Alam, Somerset, NJ (US)

Correspondence Address: KEITH D. NOWAK, CARTER LEDYARD & MILBURN LLP, 2 WALL STREET, NEW YORK, NY, 10005, US

(21) Appl. No.: 12/411,063

(22) Filed: Mar. 25, 2009

Related U.S. Application Data
(60) Provisional application No. 61/039,322, filed on Mar. 25, 2008.

(51) Int. Cl. A61B 8/00

(52) U.S. Cl. 600/459; 600/437

(57) ABSTRACT

The present invention describes systems and methods for creating a virtual force field. A field source may be scanned over a region of interest on an object using a scanning technique such as vector scanning or raster scanning. The techniques disclosed herein describe applying the force field over volumetric (spatial) regions that can extend beyond the dimensions of the focal region of the force-field source (e.g., acoustic, electromagnetic, or optical source). The present invention may equally apply to multiple processes linked by cause and effect that exhibit unequal time dependence.

### CROSS-REFERENCE TO RELATED APPLICATIONS

This application claims the benefit of, under 35 U.S.C. §119(e), U.S. Provisional Patent Application Ser. No. 61/039,322, filed Mar. 25, 2008, which is incorporated by reference herein in its entirety.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### FIELD OF THE INVENTION

The present invention relates to medical imaging. In particular, the present invention relates to systems and methods for creating a force field, specifically a virtual force field.

### BACKGROUND OF THE INVENTION

A field source is a physical phenomenon that gives rise to a force field. A non-limiting example of a field source is a focused ultrasound wave(s) produced by an ultrasound transducer. The force field is a vector field capable of imparting momentum. This field can be time-varying and spatially distributed.

It is an object of the present invention to scan a region of interest using a force field to control the response of the region of interest. It is an object of the present invention to scan a region of interest using a force field to understand the response of the region of interest. It is an object of the present invention to understand the magnitude of the force field when applied to tissues. It is an object of the present invention to understand the direction of the force field when applied to diffusive systems. It is an object of the present invention to create an arbitrarily shaped force field by scanning the field source over the region of interest.

### BRIEF SUMMARY OF THE INVENTION

The present invention describes systems and methods for creating a virtual force field. A field source may be scanned over a region of interest on an object using a scanning technique such as vector scanning or raster scanning. The techniques disclosed herein describe applying the force field over volumetric (spatial) regions that can extend beyond the dimensions of the focal region of the force-field source (e.g., acoustic, electromagnetic, or optical source). The present invention may equally apply to multiple processes linked by cause and effect that exhibit unequal time dependence.

### BRIEF DESCRIPTION OF THE DRAWINGS

FIG. 1 is a timing diagram illustrates a refractory system used with the present invention.

FIG. 2 shows a single-element moving transducer emitting an ultrasound beam to create a virtual field.

FIG. 3 shows two positions of a single moving transducer and its emitted ultrasound beam.

FIG. 4 is a block diagram showing components of one possible embodiment of an ultrasound embodiment of a raster scan.

FIG. 5 is a timing diagram illustrates a persistence system used with the present invention.

FIG. 6 is another timing diagram illustrates a persistence system where an object is able to respond to force field impulses during its response to an earlier impulse.

FIG. 7 shows the diagram of FIG. 6 wherein a response to a secondary impulse is additive.

FIG. 8 shows reactions of a suspension to the virtual force field

### DETAILED DESCRIPTION OF THE INVENTION

The present invention is described using a force field, a force source and an object situated in the force field. The force field is a vector field capable of imparting momentum. This field can be time-varying and spatially distributed. One non-limiting example of a force field includes a radiation force field arising from incident ultrasonic waves. However, other forms of waves may equally be used in the present invention, such as light waves.

The field source is a physical phenomenon that gives rise to the force field. A non-limiting example of a field source is a focused ultrasound wave(s) produced by an ultrasound transducer. The acoustic radiation force F is proportional to the gradient of the ultrasound intensity I; c is the speed of sound:

$$\vec{F} = \frac{1}{c} \vec{∇} I$$

Neglecting viscosity, the gradient of I is 2αƒ1 where α is the acoustic attenuation coefficient and ƒ is the ultrasound frequency. If values of attenuation are uniform, the force field is strongest in the focal region of the ultrasound beam where values of I are highest, and the only component of the force is directed along the beam axis in the direction of beam propagation with magnitude:

F=2αƒI1c

Accordingly, the focal region is the local force-field maximum. A further discussion of radiation-force fields can be found in a paper by Lizzi F. L., Muratore R, Deng C. X., Ketterling J. A., Alam S. K, Mikaelian S., et al. Radiation-force technique to monitor lesions during ultrasonic therapy. Ultrasound in Medicine and Biology. 2003 Nov. 29 (11):1593-605.

The object can be anything that experiences a change in momentum when subject to the force field. Non-limiting examples of objects include fluids and biological tissue. Because the force field can vary spatially, so can the object response. If the object is a fluid, then the regions near the field maxima will be expected to stream, while those far away will not. If the object is neural or cardiac tissue, the regions near the field maxima will respond, while those far away will not. The response from the object does not need to be homogeneous.

The response from the object may range from a change in momentum to electrical activity of neurons or cardiac cells when mechanically stimulated by a radiation force. When attention is being focused on the electrical activity of cells, the direction of the force field is of lesser interest than the magnitude of the force field.

Timing with Refraction

The present invention may be described using a refractory system. An example describing this embodiment uses cortical structures in the brain as objects of interest. For the sake of the present example, the behavior of the constituent neurons can be idealized as a binary response and a refractory period, though actual neuronal behavior is more complicated.

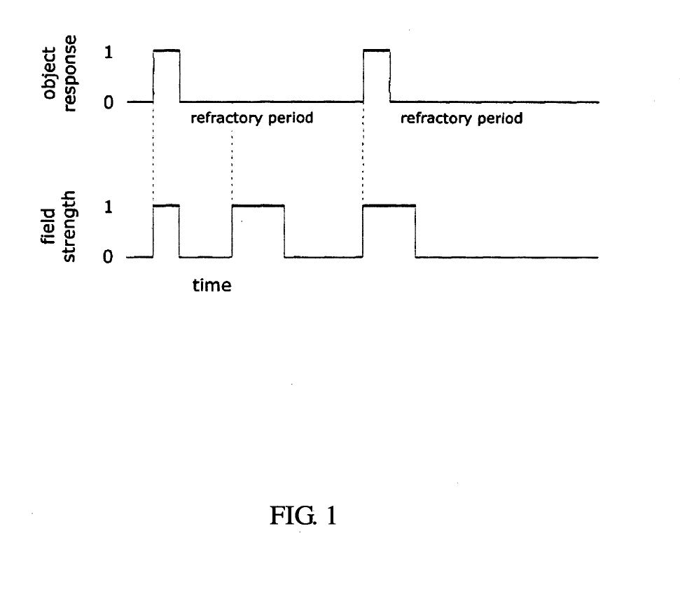

The timing diagram of FIG. 1 shows an ideal object in a force field where the local field strength varies in time.

The horizontal axis represents time increasing from left to right, and the vertical axis represents the field strength or object response in arbitrary units. The first field burst elicits a response from the object. The ideal object then undergoes a period of refraction during which it is insensitive to the field, i.e., the object does not respond to the force field during the refractory period. Once the refractory period passes, the object can once again respond to the field. Two important consequences of a refractory period are stated as the following principles:

Principle 1: During the refractory period the source is free to address a distant region without losing its influence on the local region.

Principle 2: The refractory period allows for the application of the force field in a different region close to the original site without confounding the influence upon it.

At any moment, the effective force field has a single local maximum. However, as a result of these two principles, a virtual field of arbitrary shape can be generated. The extent of the virtual field is limited by the relative timing between the ability to create a new field maximum and the refractory period. The regions in which the field is effectively influencing the object can be discontinuous. Mathematically, the sum of the field maxima over a period of overlapping refraction describes a multiply-connected domain.

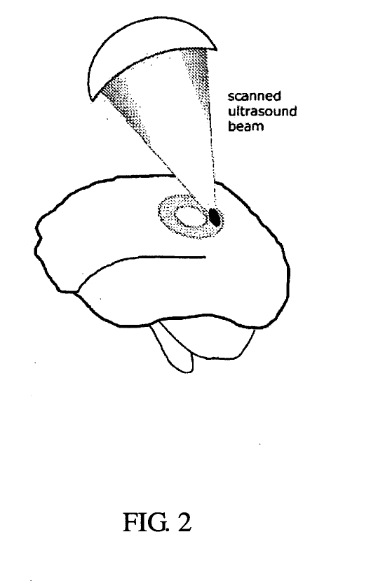

In another example, a force field is applied to experiments or treatments of the brain. Here, an ultrasound transducer generates an acoustic radiation force field on the brain, which is the object. The ultrasound beam can be modulated in intensity as the transducer orientation is manipulated and the focal region moves throughout the brain. FIG. 2 illustrates the creation of a virtual field by a single-element moving transducer shown at one position emitting an ultrasound beam. The ultrasonic source used in the present invention should not be limited to single-element transducer as other ultrasonic sources are possible including annular arrays, linear arrays, and multi-dimensional arrays. Additionally, other steering mechanisms may also be used in the present invention including array phasing.

At any particular moment, the effective force field is confined to a single local maximum. The picture above shows the single maximum as the dark ellipse at the focal region of the transducer on the right. A virtual field is created by scanning the ultrasound beam rapidly, relative to the refractory period. For example, the virtual force field can be created by moving the focal region to generate a local force field maximum at each focus and then repeating this process at different points within the overall region of interest multiple times within the refractory period. In the above picture, the virtual force field is shown extending throughout the shaded region.

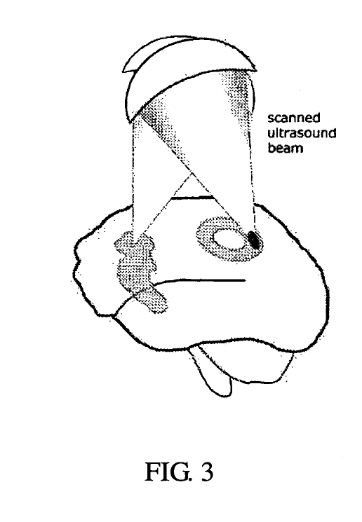

Next, a second region is added disconnected from the first. Two positions of a single moving transducer and its emitted ultrasound beam are shown in FIG. 3.

Here, a virtual field is created that extends throughout the multiply-connected region indicated by the two shaded cortical regions. Applying the field in two separate regions within the refractory period of the first insonified point can elucidate interactions between the regions. In principle, this can apply to 2, 3 or even more regions depending on refractory period duration and the rate at which local force fields can be generated.

The motion of the field source (i.e., the ultrasound focal region) can be described by analogy to imagery. One possible set of motions is a so-called vector scan, where the regions to be addressed are directly traversed. Another possible set of motions is a raster scan, where the entire volume is addressed and the tracing beam is modulated according to its position.

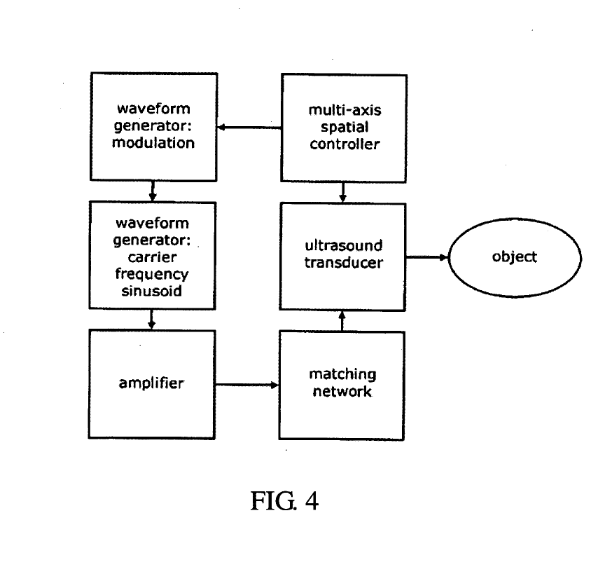

The components of one possible embodiment of the ultrasound embodiment of a raster scan are shown in the block diagram of FIG. 4.

A multi-axis spatial controller such as an xyz-positioner steers the ultrasound beam focus, for example, by moving the ultrasound transducer. The movement of the focal region may also be accomplish using a phased, one- or two-dimensional array. Simultaneously, the spatial controller provides spatial coordinates to a waveform generator. The modulation waveform generator is programmed with a look-up table or algorithm and outputs an electrical signal that depends on the spatial coordinates of the focal region. The modulation waveform generator then modulates the carrier frequency sinusoid output by a second waveform generator. The second waveform generator excites an amplifier, and the amplifier output is coupled to the transducer through an electrical impedance matching network.

The refractory system of the present invention was described using an object response and a refractory period. If the magnitude of the object response is below a threshold of interest (e.g., 0) but a refractory period ensues from the impulse, then an inhibitory system is displayed. Principles 1 and 2 apply without variation.

Timing with Persistence

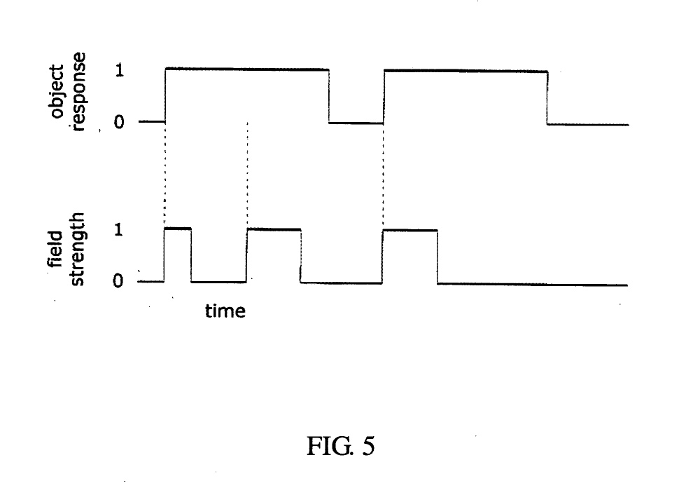

A persistence system is a system similar to the Timing with Refraction system described above and may also be used to describe the present invention. The timing diagram of FIG. 5 illustrates the persistence system of the present invention. As with the Timing with Refraction system shown above in FIG. 1, the first field burst in the persistence system elicits a response from the object. However, in this system the object responds with a response time that exceeds the stimulus time. During its response, it is insensate to the field. Therefore, the two principles apply and the consequences are the same, namely that a multiply-connected virtual field can be constructed.

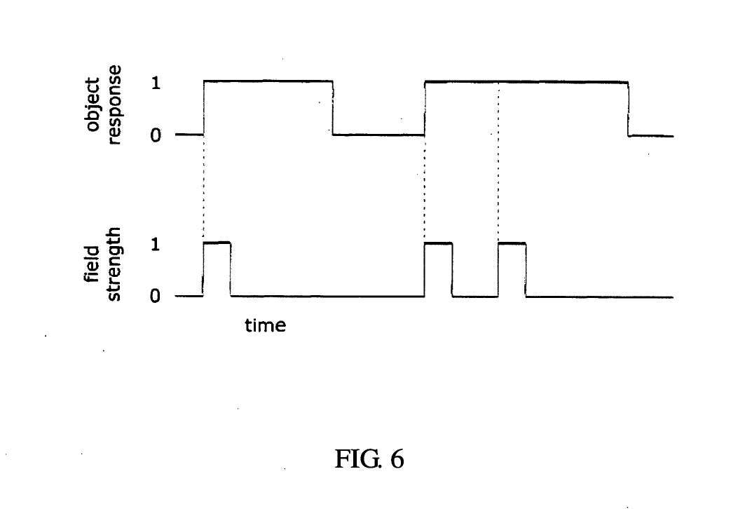

Another aspect of the persistent system describes the object as being able to respond to force field impulses during its response to an earlier impulse. The timing diagram of FIG. 6 illustrates this aspect.

Referring to the above diagram of FIG. 6, if the response to the secondary impulse simply is to increase the duration of the response, and if there is a desire to keep the object excited continuously, without interruption, then the object need only be prompted before its response has ended. Here, Principle 1 applies, but Principle 2 does not apply.

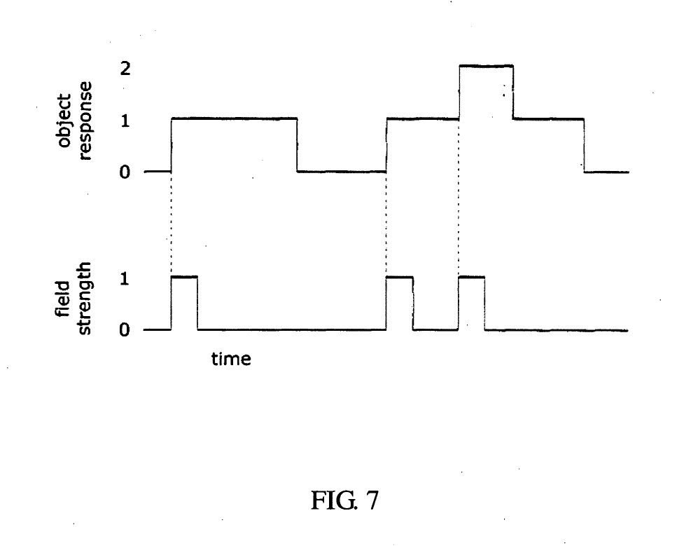

Referring now to the FIG. 7, if the response to the secondary impulse is additive, then Principle 2 does not apply. Now, however, Principle 1 is severely constrained as to maintain influence on the local object, the timing required to keep it continuously excited at a constant level will probably exceed practical limits.

If the response to the secondary impulse is subtractive, or quenches the response, then neither principle applies.

Both refractory and persistent systems of the present invention permit the creation of an extended virtual force field when the object has a characteristic time which is longer than the time needed to reposition the source and to create a new field maximum.

Timing with Diffusion

In another embodiment, the present invention may employ a diffusion system. Here, the characteristic time of the object is due to diffusion. The present system may be described in the following example using a liquid object that includes small particles suspended in the liquid. Once an impulse is applied to a small bolus at the field maximum, the suspended particles will experience a force directed along the ultrasound beam axis.

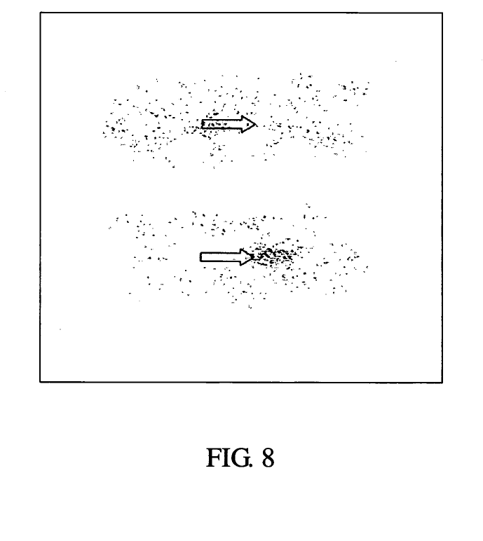

For the sake of simplicity, the present example neglects actual particle motion, as it is complicated, and the fact that the liquid can be streamed by the impulse. It should be noted that the simplification is justified because the motion of the suspended particles arises from the motion of the fluid in which they are suspended and from the impulse imparted directly to them by the radiation force. Fluid streaming arising from radiation force has been extensively discussed in the scientific literature. The present invention is confined to exposing the unique reaction of the suspension to the virtual force field. It should also be noted that in important applications where streaming is minimized, for instance, flow in a capillary vessel, flow in a porous tissue, there is greater attenuation of the beam by the suspension than by the fluid. See FIG. 8

When the impulse is removed, there will be a slightly higher concentration of suspended particles distal to the focal region, and the particles in the more-highly concentrated region will have a net momentum along the direction of the applied force. Scattering will gradually reduce the net momentum of the bolus and will lead to diffusion toward the lower concentration region where the field maximum was located. Therefore, the particles will slowly return to their original uniform concentration, with the only long-term effect being a slight overall change in particle velocity throughout the fluid.

If a second impulse is applied at the site of the first impulse before equilibrium has been restored the suspended particles more farther along the desired path. This effect is contingent on positioning the second maximum slightly toward the centroid of the peak concentration.

Accordingly, Principle 1 applies to the diffusion system as it allows for control to be maintained of a region of interest without spending much time there. Principle 2 is also applicable albeit in a weakened form. Principle 2 allows the force field to be applied close to the original site without undue influence on it.

If the goal is to guide the suspended particles in a particular direction, then it does not matter if the first and second maxima overlap. The second maximum might be located in a region that overlaps the transition from the depleted site of the first maximum and the concentration peak of the bolus from the first impulse.

The system of timing with diffusion of the present application may be useful in separating suspended particulates in situ. Components of a heterogeneous suspension diffuse at different rates. More massive or larger particles will diffuse slower than less massive or smaller particles (ignoring charge, etc.). A virtual force field can be created in a shape that will shepherd suspended particles. For example, a force field can be used to create a hemispherical or cup-shaped field that will gather particles towards the axis of rotation of the hemisphere. The lighter particles will move with each impulse. By repeatedly pulsing the field, the concentration of the lighter particles can be built up in a region of interest. In an embodiment where filters are added to the system, the force field can be used to push the suspension through the filters. This embodiment could be useful in situations when gravity or centrifugation is not available or appropriate.

In the preceding discussions of the virtual force field, the magnitude of the field was of primary importance when considering the response of objects such as neural and cardiac tissue. The direction of the force field was important also when considering diffusive systems.

General Systems

The concept of an extended virtual force field described in the present invention can be applied to any phenomenon where two linked processes exist with distinct timescales. The slower process is called p₀ and the faster process p₁. A causal relationship exists for p₁, upon p₀. The causal chain can be of various lengths, e.g., there can be intermediaries between p₁, and p₀. An example of an intermediary is a field. The field will vary in time and space.

If p₁ is considered to respond discontinuously to the field, the discontinuity may be due to a time-varying receptivity of p₁. One form of time-varying receptivity is a refractory period. If the discontinuity is due to a time-varying receptivity of p₁, then a virtual field can be created.

The limit of very short refractory periods is a continuous response. If the reaction of the process p₁, is slower than the speed with which the field can be made to vary, then a virtual field can be created.

From this general systems embodiment, applications are envisioned in which the virtual field represents the reactions of engineered components distributed throughout a network, with built-in delays.

While the invention has been described by way of example and in terms of specific embodiments it is not so limited and is intended to cover various modifications as would be apparent to those skilled in this art area. Therefore, the scope of the appended claims should be accorded the broadest interpretation so as to encompass all such modifications.

1. A method of creating a virtual force field comprising the steps of:
applying a force field to a first region on an object creating a first focal region/maximum, said application causing an object response followed by a refractory periods; and
applying said force field to a second region of said object during said refractory period creating a second focal region/maximum.

2. The method of claim 1, wherein said subsequent region is a distant region from the first region, said object response being maintained during creation of said second focal region/maximum.

3. The method of claim 1, wherein said second region is a proximal region to the first region, said object response being uninfluenced by said second focal region/maximum.

4. The method of claim 1, wherein said first focal region/maximum and said second focal region/maximum create said virtual force field, said virtual force field having an arbitrary shape.

5. The method of claim 1, wherein said first focal region/maximum is discontinuous to said second focal region/maximum.

6. The method of claim 1, wherein said virtual force field is limited by timing between ability to create said force field maximum and said refractory period.

7. The method of claim 1, wherein adding said second focal region/maximum and said subsequent focal region/maximum created during said refractory period with said first focal region/maximum forms a multiply connected virtual force field.

8. The method of claim 1, further comprising multiply re-applying said force field to different regions of said object during said refractory period creating said virtual force field having an arbitrary shape.

9. The method of claim 1, wherein said refractory period permits the creation of multiple subsequent focal region/maximum.

10. The method of claim 1, wherein said field source scans using a vector scan technique.

11. The method of claim 1, wherein said field source scans using a raster scan technique.

12. An ultrasound transducer comprising:
a multi-axis spatial controller, said controller steering an ultrasound beam, said controller generating spatial coordinates of a focal region;
a modulation waveform generator, said modulation waveform generator receiving said spatial coordinates from said controller, said modulation waveform generator having a look-up table to output an electrical signal that depends on the spatial coordinates of the focal region;
a second waveform generator, said second generator outputting a carrier frequency sinusoid, said sinusoid output being modulated by said modulation generator;
an amplifier, said amplifier being excited by said second generator to release an amplifier output; and
an electrical impedance matching network, said network coupling said amplifier output to said ultrasound transducer.

13. The transducer of claim 12, wherein said spatial controller is an xyz-positioner

14. The transducer of claim 12, wherein said focal region being moved using a phased, one dimensional array.

15. The transducer of claim 12, wherein said focal region being moved using a phased, two-dimensional array.

16. A method of creating a virtual force field comprising the steps of:
providing a liquid object, said liquid object having particles suspended therein;
applying an ultrasound beam to the liquid object creating a first focal region; and
moving said particles along a path away from the first focal region, in response to the ultrasound beam.

17. The method of claim 16, further comprising applying a second ultrasound to the liquid object creating a second focal region.

18. The method of claim 17, wherein the second ultrasound is applied at the first focal region before equilibrium is restored to said particles.

19. The method of claim 17, wherein the second focal region overlaps with the first focal region moving said particles along said path.

20. The method of claim 17, wherein the second focal region and said first focal region move said particles along a hemispherical path.
